(19) United States
(12) Patent Application Publication
(10) Pub. No.: US 2024/0000709 A1
(43) Pub. Date: Jan. 4, 2024

(54) SOL-GEL CANNABINOID FORMULATION AND ANTIVIRAL USE

(71) Applicant: PREVECEUTICAL MEDICAL INC., Vancouver (BC)

(72) Inventor: Stephen Van DEVENTER, Vancouver (BC)

(21) Appl. No.: 18/253,573

(22) PCT Filed: Nov. 19, 2021
(86) PCT No.: PCT/AU2021/051383

§ 371 Date: May 18, 2023

(30) Foreign Application Priority Data
Nov. 20, 2020 (AU) 2020904291

(51) Int. Cl. A61K 9/06; A61K 47/34; A61K 31/00; A61K 9/00

(52) U.S. Cl. CPC A61K 9/06; A61K 47/34; A61K 31/658; A61K 9/0043

(57) ABSTRACT

An antiviral cannabinoid-containing thermoresponsive sol-gel formulation and its use in the treatment or prevention of viruses such as Coronaviruses. In particular embodiments, the cannabinoid containing thermoresponsive sol-gel formulation is adapted for nasal delivery and comprises a micellar composition comprising a cannabinoid composition having at least 70% cannabidiol (CBD).

### FIELD OF INVENTION

The present invention relates to an antiviral cannabinoid-containing sol-gel formulation and its use in the treatment or prevention of viruses such as Coronaviruses. In particular embodiments, the cannabinoid containing sol-gel formulation is adapted for nasal delivery and comprises a micellar composition comprising a cannabinoid composition having at least 70% cannabidiol (CBD).

### BACKGROUND OF INVENTION

In recent years, phytocannabinoids have attracted interest across scientific and medical communities based on their purported role in the prevention and treatment of a host of ailments/diseases [Flores-Sanchez and Ramos-Valdivia, 2017]. Significant efforts have focused on the identification and isolation of phytocannabinoids from Cannabis sativa, although Cannabis indica and Cannabis ruderalis have also received some, albeit lesser attention [Hanus, et al., 2016]. Among 500 or more active chemicals present in the Cannabis plant, the two most abundant and therapeutically relevant compounds are (−)-trans-Δ⁹-tetrahydrocannabinol (Δ⁹-THC) and (−)-cannabidiol (CBD) [ElSohly, et al. 2017]. Δ⁹-THC possesses psychotropic effects mediated by the activation of CB1 receptors in the brain, while CBD has no direct agonistic action at the endocannabinoid receptor [Mackie, 2008, Zou and Kumar, 2018]. Therefore, CBD is considered non-psychotropic, although it is psychoactive, and is clinically used as a neuroprotectant in resistant seizure disorders, including Lennox-Gastaut and Dravet syndromes and in association with Δ⁹-THC is formulated for the treatment of neuropathic pain [De Vita, et al., 2018, Andreae, et al., 2015, Koppel, et al., 2014]. Approved products containing one or more plant-derived, or synthetically manufactured cannabinoids are currently available by prescription for oral administration.

In contrast to the aforementioned clinical conditions, medicinal Cannabis has also attracted attention in the context of Coronavirus (SARS-CoV-2, COVID-19) related infection. COVID-19 is responsible for a pandemic that has spread to more than 210 countries, with more than 37 million people infected and over a million deaths have been recorded, since the pandemic was first declared [https://coronavirus.jhu.edu/map.html]. No pharmacological agent is available yet to bring the virus under control and to curb its devastating effects. However, various anti-viral and anti-inflammatory drugs have been trialled with varying degrees of success. However, it has been reported that CBD has an effect in treating or preventing Covid-19 and symptoms associated with Covid-19 (https://clinicaltrials.gov/ct2/show/NCT04467918?term=cbd%2C+covid&draw=2&rank=1, https://clinicaltrials.gov/ct2/show/NCT04504877?term=cbd%2C+covid&draw=2&rank=2, https://clinicaltrials.gov/ct2/show/NCT03944447?term=cbd%2C+covid&draw=2&rank=3).

There is a need for therapies for Coronaviruses that are readily delivered to the respiratory system. The present invention is predicated at least in part on the formulation of a CBD-containing sol-gel formulation that is more patient and practitioner friendly, including formulations suitable for intranasal transmucosal delivery.

### SUMMARY OF THE INVENTION

In a first aspect of the invention, there is provided a thermoresponsive sol-gel formulation comprising:
a) a thermoresponsive poloxamer and/or poloxamine, and
b) a micellar composition comprising a plurality of micelles, said micelles comprising:

i) thermoresponsive poloxamer and/or poloxamine;

ii) surfactant in an amount of 7-9% w/w of the sol-gel formulation, wherein the surfactant is not a poloxamer and/or poloxamine; and

iii) a cannabinoid composition in an amount of 0.5 to 5 w/w of the sol-gel formulation;

wherein the sol-gel formulation has total amount of thermoresponsive poloxamer and/or poloxamine of 12 to 13.5% w/w of the sol-gel formulation, a gelation temperature between 28° C. and 32° C. and the cannabinoid composition comprises at least 70% w/w of cannabidiol.

In another aspect the present invention provides a method of treating or preventing a Coronavirus infection comprising administering a sol-gel formulation of the invention.

In yet another aspect of the present invention, there is provided a method of alleviating one or more symptoms of a Coronavirus infection comprising administering a sol-gel formulation of the invention.

In a further aspect of the invention there is provided a use of a sol-gel formulation of the invention in the manufacture of a medicament of treating or preventing a Coronavirus infection.

In yet a further aspect of the invention there is provided a use of a sol-gel formulation of the invention in the manufacture of a medicament for alleviating one or more symptoms of a Coronavirus infection.

In another aspect of the invention there is provided a sol-gel formulation of the invention for use in treating or preventing a Coronavirus infection.

In yet another aspect of the present invention there is provided a sol-gel formulation of the invention for use in alleviating one or more symptoms of a Coronavirus infection.

### DETAILED DESCRIPTION OF INVENTION

### Definitions

Unless defined otherwise, all technical and scientific terms used herein have the same meaning as commonly understood by those of ordinary skill in the art to which the invention belongs. Although any methods and materials similar or equivalent to those described herein can be used in the practice or testing of the present invention, preferred methods and materials are described. For the purposes of the present invention, the following terms are defined below.

The articles “a” and “an” are used herein to refer to one or to more than one (i.e. to at least one) of the grammatical object of the article. By way of example, “an element” means one element or more than one element.

As used herein, the term “about” refers to a quantity, level, value, dimension, size, or amount that varies by as much as 30%, 25%, 20%, 15%, 10% or 5% to a reference quantity, level, value, dimension, size, or amount.

Throughout this specification, unless the context requires otherwise, the words “comprise”, “comprises” and “comprising” will be understood to imply the inclusion of a stated step or element or group of steps or elements but not the exclusion of any other step or element or group of steps or elements.

As used herein, the term “micelle” refers to a supramolecular assembly of molecules wherein hydrophobic portions of these molecules comprise the interior of the assembled micelle (i.e., the hydrophobic polymeric core) and hydrophilic portions of the molecules comprise the exterior of the assembled micelle (i.e., the outer hydrophilic polymeric layer). In this regard, micelles are spontaneously formed by amphiphilic compounds in water above a critical solute concentration, the critical micellar concentration (CMC), and at solution temperatures above the critical micellar temperature (CMT). There are many ways to determine CMC, including surface tension measurements, solubilization of water insoluble dye, or a fluorescent probe, conductivity measurements, light scattering, and the like.

The term “gel” as used herein refers to the physical properties of the compositions of the invention, which are generally semi-solid systems that include a liquid or liquid-like component and optionally solid particles and other components, such as carrier micelles, dispersed or disposed therein. A “gel” refers to the state of matter between liquid and solid. As such, a “gel” has some of the properties of a liquid (i.e., the shape is resilient and deformable) and some of the properties of a solid (i.e., the shape is discrete enough to maintain three dimensions on a two-dimensional surface).

The term “thermoresponsive sol-gel”, as generally used herein, refers to a composition, which undergoes a phase transition from a solution or liquid phase to a gel phase (e.g., the conversion of a liquid or flowable form with a viscosity of about 0.05 Pascal-seconds or less, to a gel or relatively semi-solid form with a viscosity of at least about 0.4 Pascal-seconds) or vice versa when the temperature is raised above or reduced below a critical value, which is referred to herein as a “gelation temperature” or “transition temperature”. Preferably the phase transition from a liquid to a gel and vice versa occurs in less than 10 minutes (e.g., 5 sec, 10 sec, 15 sec, 30 sec, 1 min, 2 min, 3 min, 4 min, 5 min, 6 min, 7 min, 8 min, 9 min, 10 min and any range therein), more particularly in less than 5 minutes and even more particularly in less than 2 minutes or less than 1 minute.

It is to be understood that, if any prior art publication is referred to herein, such reference does not constitute an admission that the publication forms a part of the common general knowledge in the art, in Australia or any other country.

### Thermoresponsive Sol-Gel Formulation

The thermoresponsive sol-gel formulation of the present invention comprises a) a thermoresponsive poloxamer and/or poloxamine and b) a micellar composition. The micellar composition is a carrier composition for the cannabinoid composition to be dispersed or solubilised in an aqueous solution in a sol-gel.

The cannabinoid composition is a CBD rich cannabinoid composition where the CBD is present in an amount of at least 70% w/w, or at least 75%w/w of the cannabinoid composition. In some embodiments, the cannabinoid composition comprises 70 to 100% w/w CBD, 70 to 95% w/w CBD, 70 to 90% w/w CBD 75 to 90% w/w CBD or 75 to 85% w/w CBD. In particular embodiments, the CBD is present in an amount of about 80% w/w of the cannabinoid composition.

The cannabinoid composition may be a natural extract obtained directly by extraction of a Cannabis plant or part of a Cannabis plant, an extract that has been treated, for example by heat treatment to cause decarboxylation of at least some cannabinoids in the Cannabis extract or a composition comprising one or more isolated or synthetic cannabinoids and optionally other components. In some embodiments, the extract may be supplemented with isolated cannabinoids such as CBD to obtain the desired amount of CBD in the cannabinoid composition.

The Cannabis plant may be any species of Cannabis plant, including Cannabis sativa Linnaeus, Cannabis indica Lam., and Cannabis ruderalis as well as hybrid combinations thereof and hemp plants. Cannabis varieties may also be bred to have varying cannabinoid profiles, for example, high CBD. As used herein, the term “Cannabis” refers to any and all of these plant varieties.

Extracts of Cannabis may be prepared by any means known in the art. The extracts may be formed from any part of the Cannabis plant, for example, from leaf, seed, trichome, flower, keif, shake, bud, stem, root or a combination thereof. In some embodiments, the extract is formed from the flowers and shake of a Cannabis plant. Any suitable extractant known in the art may be used, including, for example, alcohols (e.g. methanol, ethanol, propanol, butanol, propylene glycol etc.), water, hydrocarbons (e.g. butane, hexane, etc.), oils (e.g. olive oil, vegetable oil, essential oil, etc.), a polar organic solvent (e.g. ethyl acetate, polyethylene glycol, etc.) or a sub/supercritical fluid (e.g. liquid CO₂). The extractant may be completely or partially removed prior to incorporation of the Cannabis extract into the micellar composition. In particular embodiments, any extractant may be removed by heating the extract optionally under reduced pressure (e.g. under vacuum). In some embodiments, the extract is filtered to remove particulate material, for example, by passing the extract through filter paper or a fine sieve (e.g. a sieve with pore sizes of 5 μm).

In some embodiments, the Cannabis extract is formed by applying heat and pressure to the plant material. Typically, in these embodiments, no extractant is required.

The cannabinoid fraction typically accounts for the majority of the compounds present in the Cannabis extract. In some embodiments, the Cannabis extract may comprise at least 70% w/w cannabinoids, for example, about 70% to about 99%, about 75% to about 95% or about 75% to about 90% by weight of the Cannabis extract. Other non-cannabinoid compounds that may be present include, but are not limited to, terpenes and terpenoid compounds.

In some embodiments, the cannabinoid composition is a Cannabis extract. In other embodiments, the cannabinoid composition is a non-natural composition in which a Cannabis extract has been supplemented with isolated or synthetic CBD. In yet other embodiments, the cannabinoid composition substantially consists of isolated CBD.

Cannabinoids that have been identified in Cannabis plants include: Cannabigerol (CBG), Cannabigerolic acid (CBGA), Cannabigerovarin, Cannabigerovarinic acid, (±)-Cannabichromene (CBC), (±)-Cannabichromenic acid, (±)-Cannabivarichromene, (±)-Cannabichromevarin, (±)-Cannabichromevarinic acid A, Cannabidiol (CBD), Cannabidivarin, Cannabidiorcol, Cannabidiolic acid (CBDA), Cannabidivarinic acid, Cannabinodiol, Cannabinodivarin, Δ⁹-Tetrahydrocannabinol (Δ⁹-THC/THC), Δ⁹-Tetrahydrocannabivarin (THCV), Δ⁹-Tetrahydrocannabiorcol, Δ⁹-Tetrahydrocannabinolic acid (THCA), Δ⁹-Tetrahydro-cannabivarinic acid, Δ⁹-Tetrahydrocannabiorcolic acid A and/or B, Cannabinol (CBN), Cannabivarin, Cannabiorcol, Cannabinolic acid A, Cannabitriol, Cannabiripsol, Cannabitetrol, Cannabielsoin, Cannabielsoic acid A, Cannabiglendol, Dehydrocannabifuran, Cannabifuran, Isotetrahydrocannabinol, Isotetrahydrocannabivarin, Cannabicyclol, Cannabicyclolic acid, Cannabicyclovarin, Cannabicitran, Cannabichromanone and Cannabicoumaronone. A comprehensive list of cannabinoids may be found in Mahmoud A. El Sohly and Waseem Gul, “Constituents of Cannabis sativa.” In Handbook of Cannabis Roger Pertwee (Ed.) Oxford University Press (2014) (ISBN: 9780199662685).

In particular embodiments, the cannabinoid composition comprises CBD and at least one of THC, CBDA, THCA, THCV, CBG, CBN, CBC, CBDV, CBGA or a mixture of two or more of these cannabinoids.

In some embodiments, the cannabinoid composition is a Cannabis extract that has been treated by heating to convert the CBDA and/or THCA to CBD and THC respectively by decarboxylation.

In some embodiments, the cannabinoid composition is present in the micellar composition in an amount in the range from about 1% to about 10% of the micellar composition, especially about 2.5 to about 6.25% of the micellar composition or about 5% of the micellar composition. The cannabinoid composition is present in an amount of about 0.5% to about 1.2% w/w of the sol-gel formulation, especially about 0.8% to about 1.2% w/w of the sol-gel formulation, more especially about 1% of the sol-gel formulation.

In some embodiments, the thermoresponsive poloxamer and/or poloxamine is a thermoresponsive poloxamer. The thermoresponsive poloxamer present in the micellar composition may be a nonionic triblock copolymer composed of a central hydrophobic chain of polyoxypropylene (poly(propylene oxide) or PPO) flanked by two hydrophilic chains of polyoxyethylene (poly(ethylene oxide) or PEO). Such poloxamers may be linear or branched and include notably tri-blocks or tetra-blocks copolymers. Exemplary poloxamers include F87, F88, F98, F108, F38, F127 (P407), L35, P84, P85, L62, L63, L64, P65, F68, L72, P75, F77, P105, L42, L43, L44, P103, P104, P105, L81, L101, L121, L122, P123, P124, P188, P237 and P338. In a particular embodiment, the poloxamer is P407.

It will further be appreciated that the nomenclature of poloxamers relates to their monomeric composition. The first two digits of a poloxamer number, multiplied by 100, gives the approximate molecular weight of the hydrophobic polyoxypropylene block. The last digit, multiplied by 10, gives the approximate weight percent of the hydrophilic polyoxyethylene content. For example, poloxamer 407 (P407) describes a polymer containing a polyoxypropylene hydrophobe of about 4,000 g/mol with a hydrophilic polyoxyethylene block content of about 70% of the total molecular weight. Most preferred poloxamers are ones that are pharmaceutically acceptable for the intended route of administration of the gel-based composition or the sol-gel composition.

It will be appreciated that a poloxamer and/or poloxamine that make up the micellar composition may be any physiologically acceptable poloxamer or poloxamine known in the art that is capable of micelle formation. Additionally, it is envisaged that the poloxamer and/or poloxamine may include a plurality (e.g., 2, 3, 4, 5 etc or more) of poloxamers and/or poloxamines respectively.

In some embodiments, the poloxamer is selected from the group consisting of P407, P124, P188, P237, P338 and any combination thereof. In particular embodiments, the poloamer is or comprises P407 (also known as F127).

The term “poloxamine” denotes a polyalkoxylated symmetrical block copolymer of ethylene diamine conforming to the general type [(PEG)ₓ-(PPG)_{γ}]₂—NCH₂CH₂N-[(PPG)_{γ}-(PEG)_{χ}]₂. Each poloxamine name is followed by an arbitrary code number, which is related to the average numerical values of the respective monomer units denoted by X and Y. Poloxamines are typically prepared from an ethylene diamine initiator and synthesized using the same sequential order of addition of alkylene oxides as used to synthesize poloxamers. Structurally, the poloxamines generally include four alkylene oxide chains and two tertiary nitrogen atoms, at least one of which is capable of forming a quaternary salt. Poloxamines are usually also terminated by primary hydroxyl groups.

Poloxamines are commercially available in a wide range of EO/PO (ethyleneoxide (EO)/propylene oxide (PO)) ratios and molecular weights under the tradename Tetronic® (BASF). Exemplary poloxamines include T304, T701, T707, T901, T904, T908, T1107, T1301, T1304, T1307, T90R4, T150R1 and T1508, whose properties are shown in the table below.

| | | EO units per | PO units per | |
|---|---|---|---|---|
| Tetronic | Mw (Da) | block (a) | block (b) | HLB |
| 304 | 1650 | 3.7 | 4.3 | 12-18 |
| 701 | 3600 | 2.1 | 14.0 | 1-7 |
| 901 | 4700 | 2.7 | 18.2 | 1-7 |
| 904 | 6700 | 15 | 17 | 12-18 |
| 908 | 25000 | 114 | 21 | >24 |
| 1107 | 15000 | 60 | 20 | 18-23 |
| 1301 | 6800 | 4 | 26 | 1-7 |
| 1304 | 10500 | 21.4 | 27.1 | 12-18 |
| 1307 | 18000 | 72 | 23 | >24 |
| 90R4 | 6900 | 16 | 18 | 1-7 |
| 150R1 | 8000 | 5 | 30 | 1-7 |

As used herein, the term “block copolymer” can refer to a polymer in which adjacent polymer segments or blocks are different, i.e., each block comprises a unit derived from a different characteristic species of monomer or has a different composition of units.

In particular embodiments, the poloxamer and/or poloxamine has a molecular weight of between about 1,000 to about 20,000 (e.g., about 1000, 1500, 2000, 2500, 3000, 3500, 4000, 4500, 5000, 5500, 6000, 6500, 7000, 7500, 8000, 8500, 9000, 9500, 10000, 10500, 11000, 11500, 12000, 12500, 13000, 13500, 14000, 14500, 15000, 15500, 16000, 16500, 17000, 17500, 18000, 18500, 19000, 19500, 20000 and any range therein).

In certain embodiments, the poloxamer and/or poloxamine have a ratio EO units per block to PO units per block of between about 6:1 to about 1:6 (e.g., about 6:1, 5.5:1, 5:1, 4.5:1, 4:1, 3.5:1, 3:1, 2.5:1, 2:1, 1.5:1, 1:1, 1:1.5, 1:2, 1:2.5, 1:3, 1:3.5, 1:4, 1:4.5, 1:5, 1:5.5, 1:6 and any range therein).

The poloxamer and/or poloxamine may be present in the micellar composition an amount from about 5% to about 98.5% or any range therein such as, but not limited to, about 10% to about 97%, about 20% to about 95%, or about 30% to about 90% by weight of the micellar composition. In particular embodiments, the poloxamer and/or poloxamine is present in an amount of about 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 98.5% or any range therein, by weight of the carrier micelle. In particular embodiments, the poloxamer and/or poloxamine is present in an amount of about 50% to about 80% w/w of the micellar composition.

In particular embodiments, the thermoresponsive poloxamer and/or poloxamine is a thermoresponsive poloxamer.

The surfactant described herein may be any as are known in the art and is suitably selected from the group consisting of a polyoxyethylated sorbitan fatty ester (i.e., a polysorbate—e.g., Tween® 20 (polyoxyethylene sorbitan monolaurate), Tween® 40 (polyoxyethylene sorbitan monopalmitate), Tween® 60 (polyoxyethylene sorbitan monostearate), Tween® 80 (polyoxyethylene sorbitan monooleate), a polyoxyethylated glycol monoether (e.g., macrogol 15 hydroxystearate, polyethylene glycol (15)-hydroxystearate, polyoxyethylated 12-hydroxystearic acid (Kolliphor® HS15, Solutol®)), a polyoxyethylated glyceride, n-dodecyl tetra (ethylene oxide), a polyoxyethylated fatty acid, a polyoxyethylated castor oil (e.g. Cremophor® EL (CrEL) or Kolliphor® EL), a sucrose ester, a lauroyl macroglyceride, a polyglycolyzed glyceride and combinations thereof. In some embodiments, the surfactant comprises one or more C₁₂-C₂₆ alkene, diene or polyene, for example, a surfactant comprising a fatty acid selected from one or more of myristoleic acid, palmitoleic acid, sapienic acid, oleic acid, elaidic acid, vaccenic acid, linoleic acid, linoelaidic acid, α-linolenic acid, arachidonic acid, eicosapentaenoic acid, erucic acid and docosahexaenoic acid, especially where one or more alkenes are in the cis configuration such as myristoleic acid, palmitoleic acid, sapienic acid, oleic acid, linoleic acid, α-linolenic acid, arachidonic acid, eicosapentaenoic acid, erucic acid and docosahexaenoic acid.

In some embodiments, the surfactant is a non-ionic surfactant. In some embodiments, the surfactant is or comprises a polyoxyethylene sorbitan C₁₅-₂₁ alkene, especially a polyoxyethylene sorbitan C₁₅-C₂₁ alkene with one or more double bonds in the cis configuration. An exemplary surfactant is polyoxyethylene (20) sorbitan monooleate (e.g., Tween® 80). In particular embodiments, the surfactant is a liquid at room temperature (e.g., at about 20° C. to about 25° C.). Without intending to be limited by theory, it is believed that the surfactant with its amphiphilic structure is responsible for further stabilising the cannabinoid-filled carrier micelles by associating with complementary components of the poloxamer and/or poloxamine and enhancing the retention of the cannabinoid composition in the micelles when dispersed or disposed in the sol-gel formulation, so as to improve stability and efficacy thereof.

In some embodiments, the surfactant is present in an amount of about 1% to about 50% (e.g., about 1.0%, 1.5%, 2.0%, 2.5%, 3.0%, 3.5%, 4.0%, 4.5%, 5.0%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%), or any range therein, by weight of the micellar composition, and in some embodiments from about 5% to about 45% or from about 20% to about 45% or from about 30 to 45% surfactant by weight of the micellar composition. In some embodiments, increasing the surfactant content can delay or increase the transition or gelation time of the sol-gel formulation in which the micellar composition is incorporated, whilst decreasing the surfactant content can decrease the transition or gelation time of the sol-gel formulation in which the micellar composition is incorporated. In other embodiments, decreasing the surfactant content will decrease the transition temperature of the sol-gel formulation in which the micellar composition is incorporated. Alternatively, increasing the surfactant content can increase the transition temperature of the sol-gel formulation in which the micellar composition is incorporated.

In some embodiments, the surfactant has a Hydrophile-Lipophile Balance (HLB) number between about 5 and about 20 (e.g., about 5, 6, 7, 8, 9, 10, 11, 12, 13, 14, 15, 16, 17, 18, 19, 20 and any range therein). As will be understood by the skilled artisan, HLB is a numerical system used to describe the relationship between the water-soluble and oil-soluble parts of a nonionic surfactant.

In some embodiments, the micellar composition further comprises a preservative. Suitable preservatives include parabens such as propyl paraben or methyl paraben. In particular embodiments, the micellar composition comprises propyl paraben. In some embodiments, the micellar composition is free of preservative. When present, the preservative may be present in an amount of about 0.01 to about 1.0% w/w of the micellar composition, especially about 0.15% of the micellar composition, or about 0.01 to about 0.1% w/w or about 0.03% w/w of the sol-gel formulation.

The micellar composition may be made by preparing an aqueous solution of poloxamer and/or poloxamine. The cannabinoid composition may be prepared in a water miscible solvent for example, an alcoholic solution such as ethanol, to which is added the surfactant and the composition mixed to form a uniform mixture. The poloxamer is then mixed into the cannabinoid mixture with gentle stirring during which the poloxamer and surfactant self-assemble into a micelle encapsulating the cannabinoid composition. The water miscible solvent may then be removed, for example, in vacuo. The resultant aqueous solution of micelles may then be frozen, for example, in liquid nitrogen, and the resulting mixture lyophilised for a period of time suitable to provide a dry powder micellar composition comprising the cannabinoid composition. The dry powder micellar composition may be stored at −20° C. until use.

In particular embodiments, the micellar composition is in dry powder form.

In particular embodiments, the micelles in the micellar composition are nanomicelles. Nanomicelles have an average size, which refers to the average diameter of the micelle, that may be, for example, no greater than 1000 nanometers, no greater than 500 nanometers, no greater than 200 nanometers, no greater than 100 nanometers, no greater than 75 nanometers, no greater than 50 nanometers, no greater than 40 nanometers, no greater than 25 nanometers, or no greater than 20 nanometers. In certain embodiments, the carrier micelle of the present invention has an average size of between about 10 nm and about 500 nm, or any range therein such as, but not limited to, about 15 nm to about 400 nm, or about 20 nm to about 250 nm. In particular embodiments of the present invention, the carrier micelle has an average size of about 10 nm, 20 nm, 30 nm, 40 nm, 50 nm, 60 nm, 70 nm, 80 nm, 90 nm, 100 nm, 110 nm, 120 nm, 130 nm, 140 nm, 150 nm, 160 nm, 170 nm, 180 nm, 190 nm, 200 nm, 210 nm, 220 nm, 230 nm, 240 nm, 250 nm, 260 nm, 270 nm, 280 nm, 290 nm, 300 nm, 310 nm, 320 nm, 330 nm, 340 nm, 350 nm, 360 nm, 370 nm, 380 nm, 390 nm, 400 nm, 410 nm, 420 nm, 430 nm, 440 nm, 450 nm, 460 nm, 470 nm, 480 nm, 490 nm, 500 nm or any range therein. In particular embodiments of the present invention, the micelles in the micellar composition have an average size of between about 10 nm and about 35 nm.

In particular embodiments, the micelles, when dispersed in aqueous solution have a turbidity within the range of 1 to 10 NTU.

The thermoresponsive sol-gel formulation comprises a) a thermoresponsive poloxamer and/or poloxamine and b) a plurality of micelles. The plurality of micelles is a micellar composition as described above. In some embodiments, the plurality of micelles or micellar composition is present in the sol-gel formulation in an amount of about 10% to about 35% w/w of the sol-gel formulation, especially about 15% to about 25% w/w of the sol-gel formulation. In some embodiments, the cannabinoid composition is present in an amount of between about 0.5% and 5% w/w of the total sol-gel formulation, especially about 0.5 to 2% w/w or 0.5 to 1.5% w/w of the total sol-gel formulation, more especially about 1% w/w of the sol-gel formulation. In some embodiments, the surfactant in the micellar composition is present in an amount of about 7% to about 9% w/w of the sol-gel formulation, especially about 8% w/w of the sol-gel formulation.

The thermoresponsive poloxamer and/or poloxamine in part a) of the sol-gel formulation is any poloxamer and/or poloxamine as defined above that has thermo-responsive properties such that in an aqueous solution, the poloxamer and/or poloxamine undergoing a phase transition from a solution or liquid phase to a gel phase (e.g., the conversion of a liquid or flowable form with a viscosity of about 0.05 Pascal-seconds or less, to a gel or relatively semi-solid form with a viscosity of at least about 0.4 Pascal-seconds) or vice versa when the temperature is raised above or reduced below a critical value, which is referred to herein as a “gelation temperature” or “transition temperature”. Preferably the phase transition from a liquid to a gel and vice versa occurs in less than 10 minutes (e.g., 5 sec, 10 sec, 15 sec, 30 sec, 1 min, 2 min, 3 min, 4 min, 5 min, 6 min, 7 min, 8 min, 9 min, 10 min and any range therein), more particularly in less than 5 minutes and even more particularly in less than 2 minutes and most particularly less than 1 minute.

The thermoresponsive poloxamer and/or poloxamine may be selected from the poloxamers described above for use in the formation of the micellar composition. In some embodiments, the poloxamer and/or poloxamine used in the formation of the sol-gel formulation is the same as the poloxamer and/or poloxamine used in the formation of the micellar composition. In other embodiments, the poloxamer and/or poloxamine used in the formation of the sol-gel formulation is different from the poloxamer and/or poloxamine used in the formation of the micellar composition.

The poloxamer and/or poloxamine in part a) of the sol-gel formulation (excluding any poloxamer and/or poloxamine in the micellar composition) may be present in an amount from about 0.5% to about 10% or any range therein such as, but not limited to, about 0.5% to about 5%, or about 0.5% to about 2% or about 0.7% to about 0.9% by weight of the sol-gel formulation. In particular embodiments of the present invention, the thermoresponsive poloxamer in part a) of the sol-gel formulation is present in an amount of about 0.7%, 0.8%, or 0.9% or any range therein, by weight of the sol-gel formulation.

In some embodiments, the poloxamer and/or poloxamine in the sol-gel composition is the same as the poloxamer and/or poloxamine in the micellar composition. In particular embodiments, the thermoresponsive poloxamer and/or poloxamine is a thermoresponsive poloxamer. In particular embodiments, the poloxamer in the micellar composition and in the sol-gel formulation is P407. In these embodiments, the total amount of P407 in the sol-gel formulation, including the P407 in the micellar composition, is in the range of 12 to 14% or 12 to 13.5 w/w the sol-gel formulation, especially about 12.5% to about 13.1% w/w of the sol-gel formulation, especially about 12.6% w/w to about 13% w/w of the sol-gel formulation or about 12.8% w/w of the sol-gel formulation.

In some embodiments, the sol-gel formulation further comprises a preservative. Suitable preservatives include parabens such as propyl paraben or methyl paraben. In particular embodiments, the sol-gel formulation comprises methyl paraben added in part a) of the sol-gel formulation in addition to any preservative used in the micellar composition. In some embodiments, the sol-gel formulation is free of preservative. In some embodiments, the micellar composition comprises propyl paraben as it is more hydrophobic and the sol-gel comprises methyl paraben as it is more hydrophilic. Without wishing to be bound by theory, propyl paraben may provide better protection against microbial spoilage inside the micelle where the cannabinoid resides in the hydrophobic centre of the micelle and methyl paraben may provide protection against microbial spoilage in the sol-gel water-based part of the formulation. When present in part a) of the sol-gel formulation, the preservative may be present in an amount of about 0.01 to about 1.0% w/w of the sol-gel, especially about 0.15% of the sol-gel formulation. When present, the total amount of preservative in the sol-gel formulation, including in the micellar composition, is in the range of 0.01 to 2% w/w of the sol-gel formulation, especially about 0.01 to 0.2% w/w of the sol-gel formulation.

In some embodiments, the sol-gel formulation further comprises one or more pharmaceutically acceptable components selected from a stabilising agent (polycarbophil or polyvinyl alcohol), a mechanical strength enhancer (e.g., hydroxypropyl methyl cellulose; HPMC E4M), a mucoadhesive (e.g., chitosan, polyvinyl alcohol (PVA), pentachlorophenol (PCP)) and a thickening agent/emulsifier (e.g., HPMC).

Each of the aforementioned excipients (e.g., mechanical strength enhancers, mucoadhesives, thickening agents, emulsifiers etc.) may be included in the sol-gel composition or the gel-based formulation at in an amount of about 0.05% to about 10% (e.g., about 0.05%, 0.1%, 0.2%, 0.3%, 0.4%, 0.5%, 1.0%, 1.5%, 2.0%, 2.5%, 3.0%, 3.5%, 4.0%, 4.5%, 5.0%, 6%, 7%, 8%, 9%, 10%), or any range therein, by weight of the sol-gel formulation. Generally, such excipients can be included in the solution of poloxamer and/or poloxamine when preparing the sol-gel and/or added separately to the sol-gel formulation.

In certain embodiments, the sol-gel formulation may include one or more pH-adjusting agents. For example, hydrochloric acid solutions or sodium hydroxide solutions may be added to adjust the pH of the formulation. In certain embodiments, the pH of the composition is from about 5.0 to about 8.2 (e.g., about 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2 and any range therein).

The aqueous solution may be any physiologically suitable aqueous solution including water, buffer solution, a salt solution, a saline solution, a sugar solution or a glucose solution. In particular embodiments, the aqueous solution is buffer or water, especially water.

In some embodiments, the sol-gel composition does not contain a solvent other than water. For example, in some embodiments, the sol-gel composition is substantially free or free of alcoholic solvents such as methanol, ethanol, propanol, 1,2-propanediol and the like. In some embodiments, the sol-gel composition is free of or substantially free of chromane compounds such as tocopherols. In some embodiments, the sol-gel composition is free or substantially free of cyclodextrin compounds. By “substantially free” is meant that the component is present in an amount that is less than 1%, less than 0.9%, less than 0.8%, less than 0.7%, less than 0.6%, less than 0.5%, less than 0.4%, less than 0.3%, less than 0.2%, less than 0.1%, less than 0.01%, and the like.

In particular embodiments, the thermoresponsive sol-gel formulation described herein has a viscosity of less than about 0.15 Pa·s (e.g., 0.15, 0.14, 0.13, 0.12, 0.11, 0.10, 0.09, 0.08, 0.07, 0.06, 0.05, 0.04, 0.03, 0.02, 0.01 Pa·s etc. and any range therein) at about 8° C. and greater than about 0.3 Pa·s (e.g., 0.3, 0.35, 0.4, 0.45, 0.5, 0.55, 0.6, 0.65, 0.7, 0.75, 0.8, 0.85, 0.9, 0.95, 1.0, 1.1, 1.2, 1.3, 1.4, 1.5, 1.6, 1.7, 1.8,. 1.9 Pa·s etc. and any range therein) at about 30° C. It will be appreciated that viscosity may be assessed by any means known in the art, such as with a viscometer or rheometer.

In one embodiment, the thermoresponsive sol-gel formulation has a gel strength of greater than about 500 Pa at about 30° C. and more particularly greater than about 1000 Pa at about 30° C., for example, greater than about 1000, 1050, 1100, 1150, 1200, 1250, 1300, 1350, 1400, 1450, 1500, 1550, 1600, 1650, 1700, 1750, 1800, 1850, 1900, 1950, 2000, 2050, 2100, 2200, 2250, 2300, 2350, 2400, 2450, 2500, 2550, 2600, 2650, 2700, 2750, 2800, 2850, 2900, 2950 or 3000 Pa at about 30° C. or any range there in. As used herein, the term “gel strength” refers to the rheology of the gel. These viscoelastic properties of the thermoresponsive sol-gel formulation can be determined using standard rheological characterisation techniques that will be well known to one having ordinary skill in the art.

The thermoresponsive sol-gel formulation is suitably capable of a sol-gel phase transition at a transition or gelation temperature of about 28° C. to about 32° C., especially between about 29° C. to 31° C. (e.g., about 28.0° C., 28.1° C., 28.2° C., 28.3° C., 28.4° C., 28.5° C., 28.6° C., 28.7° C., 28.8° C., 28.9° C., 29.0° C., 29.1° C., 29.2° C., 29.3° C., 29.4° C., 29.5° C., 29.6° C., 29.7° C., 29.8° C., 29.9° C. 29.0° C., 30° C., 30.1° C., 30.2° C., 30.3° C., 30.4° C., 30.5° C., 30.6° C., 30.7° C. 30.8° C., 30.9° C., 31.0° C., 31.1° C., 31.2° C., 31.3° C., 31.4° C., 31.5° C., 31.6° C., 31.7° C., 31.8° C., 31.9° C., 32.0° C.) and any range therein.

The sol-gel formulation of the present invention is preferably a solution that is substantially free of particulates or suspended matter particulates at temperatures from about 2° C. to about 30° C. and more particularly about 10° C. to about 25° C. The turbidity or clarity of the compositions of the invention may be determined by any means known in the art, such as visually and turbidimetry. Suitably, the sol-gel formulation has or demonstrates a level of turbidity or clarity of 50 Nephelometric Turbidity Units (NTU) (e.g., 50, 45, 40, 35, 30, 25, 20, 15, 10, 5 NTU or any range therein) or less, more particularly less than 20 NTU and even more particularly less than 10 NTU at temperatures from about 2° C. to about 30° C. and more particularly about 10° C. to about 25° C.

Suitably, the sol-gel formulation is a single-phase solution, at typical storage temperatures (e.g., about 2° C. to about 20° C.), but when applied to, for example, a mucosal surface of a warm blooded subject (e.g., about 25° C. to about 37° C.) the sol-gel formulation is converted to a gel that preferably possesses appropriate rheological and mechanical properties to promote retention at the site of application and ensure reproducible, sustained delivery of the therapeutic agent thereto. Possible advantages of this sol-gel formulation include enhanced drug absorption and residence time at the target site, such as a mucosal surface (e.g., the nasal mucosa), and thereby allowing for reduced dosages and dosing frequencies, reduced irritation at the site of application, improved patient compliance and the avoidance of anterior leakage and post-nasal dripping of drug for nasal applications. The sol-gel formulation may also provide a “barrier effect” that once coated on the nasal tissue, it also serves as a physical barrier, protecting the underlying mucosa from attachment of inhaled pathogens, such as viruses.

The gelation or transition temperature of the sol-gel formulation described herein may be determined by any means known in the art, such as with a rheometer or by visual inspection. To this end, it will be appreciated that visual gelation temperatures are typically higher (e.g., about 4-5° C. higher) than equivalent or corresponding rheologically determined gelation temperatures. Accordingly, in particular embodiments the gelation temperatures recited herein are rheological gelation temperatures.

The sol-gel formulations of the invention may be prepared by preparing an aqueous solution of poloxamer and/or poloxamine and an aqueous composition comprising the micellar composition described above in separate compositions. The two compositions may then be mixed to prepare the thermoresponsive sol-gel formulation.

In particular embodiments, the sol-gel formulation is suitable for nasal delivery.

In a particular embodiment, there is provided a thermoresponsive sol-gel formulation comprising
a) P407 in an amount of about 0.8% w/w of the sol-gel formulation and
b) a micellar composition comprising a plurality of micelles, said micelles comprising:

i) P407 in an amount of about 12% w/w of the sol-gel formulation;

ii) polyoxyethylene sorbitan monooleate in an amount of 7-9% w/w of the sol-gel formulation; and

iii) a cannabinoid composition in an amount of 0.5 to 1.2% w/w of the sol-gel formulation;

wherein the sol-gel formulation has total amount of P407 of about 12.8%w/w of the sol-gel formulation, a gelation temperature between 29° C. and 31° C. and the cannabinoid composition comprises at least 75% w/w of cannabidiol.

### Delivery of Sol-Gel Composition to the Nasal Epithelium

The thermoresponsive sol-gel formulation of the invention is suitable delivery to the nasal cavity, especially for sustained delivery of cannabinoids to the nasal cavity. In some embodiments, the nasal delivery is to the olfactory epithelium and underlying olfactory bulb and nerves or the trigeminal nerve network underlying the nasal epithelium. In particular embodiments, the thermoresponsive sol-gel formulation delivers the cannabinoids to the olfactory epithelium.

In some embodiments, the thermoresponsive sol-gel formulation provides sustained delivery of cannabinoids to the subject. For example, the cannabinoids may diffuse from the sol-gel formulation over a period of time, for example, hours or days. The delivery device used for the delivery of the thermoresponsive sol-gel formulation may be selected to optimise the delivery of the formulation to the nasal epithelium based on the mechanical and rheological properties of the sol-gel formulation.

Suitable nasal delivery devices include, but are not limited to, Pump 140 μL CPS (product number 31019927), Aptar Classic, Aptar Actator and Aptar gel devices from Aptar Pharma, France.

### Methods of Treatment

The thermoresponsive sol-gel formulation of the invention is suitable for treating or preventing a Coronavirus infection or a symptom of a Coronavirus infection and therefore the present invention provides a method of treating or preventing a Coronavirus infection or a symptom of a Coronavirus infection comprising administering a thermoresponsive sol-gel formulation of the invention.

Coronaviruses are viruses named for the crown-like spikes on their surfaces. There are four main subgroupings of Coronaviruses, known as alpha, beta, gamma and delta Coronaviruses. Examples of such coronaviruses include human coronavirus 229E, NL63 (HCoV-NL63), OC43, HKU1, Middle East Respiratory Syndrome MERS-CoV, severe acute respiratory syndrome coronavirus (SARS-CoV) and severe acute respiratory syndrome coronavirus 2 (SARS-CoV-2 or Covid-19). In particular embodiments, the coronavirus is SARS-CoV-2.

The symptoms of Coronavirus infection include fever, tiredness, respiratory symptoms including sore throat, dry cough, difficulty breathing or chest pain or pressure, aches and pains, diarrhea, conjunctivitis, headache or loss of sense of smell or taste. The methods and uses of the present invention may alleviate one or more of these symptoms.

The methods and uses of the present invention may further comprise administering an additional active agent useful in the treatment or prevention of Coronavirus in combination with the sol-gel formulation. Suitable active agents useful in the treatment or prevention of coronavirus include antiviral agents such as remdesivir or molnupiravir and/or anti-inflammatory agents such as dexamethasone.

The subject to which the thermoresponsive sol-gel formulation of the invention is delivered may be any subject that suffers from Coronavirus infection and to which the sol-gel formulation may be delivered. In particular embodiments, the subject is a human.

In a further aspect of the invention there is provided a use of a thermoresponsive sol-gel formulation of the invention in the manufacture of a medicament of treating or preventing a Coronavirus infection.

In yet a further aspect of the invention there is provided a use of a thermoresponsive sol-gel formulation of the invention in the manufacture of a medicament for alleviating one or more symptoms of a Coronavirus infection.

In another aspect of the invention there is provided a thermoresponsive sol-gel formulation of the invention for use in treating or preventing a Coronavirus infection.

In yet another aspect of the present invention there is provided a thermoresponsive sol-gel formulation of the invention for use in alleviating one or more symptoms of a Coronavirus infection.

### EXAMPLES

### Example 1: Preparation of a Sol-Gel Formulation

A CBD-containing cannabinoid extract was obtained from Medcan Australia and analysed to confirm the cannabinoid content. The cannabinoid extract comprised the following cannabinoids:

| | | Amount % w/w of |
|---|---|---|
| Cannabinoid | Cas No. | composition |
| Cannabidiol (CBD) | 13956-29-1 | 79.99 |
| Cannabidiol acid (CBDA) | 1244-58-2 | <0.1 |
| Δ⁹-tetrahydrocannabinol | 1972-08-3 | 2.45 |
| (Δ⁹-THC) | | |
| Δ⁹-tetrahydrocannabinolic | 23978-85-0 | <0.1 |
| acid (Δ⁹-THCA) | | |
| Cannabichromene (CBC) | 20675-51-8 | 2.28 |
| Cannabinol (CBN) | 521-35-7 | 0.29 |
| Cannabigerol (CBG) | 25654-31-3 | 1.91 |
| Cannabigerolic acid | 25555-57-1 | <0.1 |
| (CBGA) | | |
| Tetrahydrocannabivarin | 28172-17-0 | <0.1 |
| (THCV) | | |
| Cannabidivarin (CBDV) | 24274-48-4 | 0.61 |

The analysis was performed by HPLC with UV detection by Australian Cannabis Laboratories, Victoria, Australia.

Preparation of a 1% CBD-distillate-infused sol-gel followed a two-stage process, with the consecutive steps associated with each stage detailed below:

### Stage 1: Preparation of 1% w/w CBD-Distillate-Infused Micellar Powder Pre-Formulation

First, 1.2 g of poloxamer P407 (equivalent to 12% w/w of the final sol-gel) was added to a 100 mL round bottom flask (RBF), and to this 10 mL of cold water (2-8° C.) was added. This P407 mixture was stirred at 400 rpm on a magnetic stirrer for 1 hour and left to hydrate overnight at fridge temperature (2-8° C.).

Next, 0.1 g of the CBD distillate (equivalent to 1% w/w of the final sol-gel formulation) was taken in an Eppendorf tube, and 1 mL of ethanol was added. The mixture was vortexed for 10-20 seconds, after which a clear solution was attained.

The CBD ethanolic solution was added to the hydrated P407 solution and mixed by gentle hand shaking.

Next, 0.8 g of T80 (8% w/w of the sol-gel formulation) and 0.003 g of propyl paraben (PP) (0.03% w/w of the sol-gel formulation) was added to the P407-CBD ethanolic solution and the whole system was stirred for 30 minutes (400 rpm) at room temperature (22±2° C.).

Next, ethanol was removed from the mixture using a rotary evaporator (40° C. water bath temperature) with the round bottom flask rotated at 100 rpm for 30 minutes at reduced pressure (80 millibar).

The resultant aqueous solution was then frozen in the round bottom flask using liquid nitrogen, and the sample was then lyophilised for 48 hours. Lyophilised CBD distillate-infused micellar powder was generated and stored at −20° C.

### Stage 2: Preparation of 1% w/w CBD Distillate-Infused Micelle-Loaded Sol-Gel Formulation

First 0.08 g of poloxamer P407 (0.8% w/w of the sol-gel formulation) and 0.003 g of methyl paraben (MP) (0.03% w/w of the sol-gel formulation) were added to a 20 mL sample container. Next, 7.8 mL of deionised water was added to the P407-MP mixture, which was stirred for 1 hour at fridge temperature (2-8° C.) and left to hydrate overnight in the fridge.

Next, 2.10 g of CBD distillate-infused micellar powder (equivalent to 12% w/w P407, 8% w/w Tween 80 (T80) and 1% w/w CBD distillate of the final sol-gel formulation) was added to the sample container containing hydrated P407-MP mixture, in three portions. The whole system was stirred at room temperature (22±2° C.) for 15-20 minutes at 400 rpm to attain a uniform solution. Then, the whole system was transferred to a cold room (2-8° C.) and stirred (400 rpm) for a further 2 hours. After this time the stirring was stopped and the mixture was allowed to continue to hydrate in the cold room for a further 24 hours, which generated a pale yellow/straw-coloured, clear homogenous viscous solution.

The CBD distillate-infused micelle-loaded sol-gel formulation was then decanted into a suitable container, and the head space flushed with a blanket of nitrogen gas before securing the lid. The container was covered in foil and stored at fridge temperature (2-8° C.) for further evaluation.

### Example 2: Preparation of a Preservative Free Sol-Gel Formulation

The method of Example 1 was repeated but with omission of the propyl paraben in the preparation of the micelles in Step 1 and methyl paraben in the preparation of the sol-gel in Step 2.

### Example 3: Physicochemical Properties of the Micelle Compositions and the Sol-Gel Formulations

The physicochemical characteristics of the dried polymeric CBD-micelles obtained from ‘Stage 1’ were assessed for their visual appearance when dispersed in water, and resultant turbidity, micellar size and polydispersity. Polymeric CBD-micelle loaded sol-gels were assessed for their visual appearance, rheological features, and mucoadhesive properties. These evaluations provide valuable information regarding functional characteristics of the developed sol-gel and product formulation insights related to scale-up optimisation parameters, which would ultimately impact formulation performance and bioavailability of infused CBD cannabinoid formulations.

Turbidity is a measure of the cloudiness/haziness of a fluid caused by particles in suspension that cannot always be visually detected. A laser turbidometer TU5200 (Hatch Pacific-Queensland AU) determined the turbidity of developed Cannabisinfused micelle pre-formulations. In brief, dry CBD-micellar powder (50 mg) was dispersed and mixed with deionised water (10 mL) and then transferred into glass cells for direct measurement of turbidity (NTU) at room temperature. Values were recorded with standard criteria for precipitate/haze-free formulations displaying NTU values in the desired range of 1-10 NTU.

Dynamic light scattering is the most common measurement technique for particle size analysis in the nanometer range. Micellar size measurements are based on the movement of micelles in a dispersant media and the polydispersity index (PDI) describes the relative size distribution of the micelles. Briefly, a Zetasizer Nano ZS instrument (Malvern Instruments, UK) was set up for measurements at room temperature and dry micelle powder (50 mg) was dispersed in deionised water (1 mL) with aliquots placed in cuvettes. One sample at a time was placed into the holder and measured for particle size and PDI, with the average of three readings recorded for each sample.

Thermoresponsive properties of the sol-gel formulations were evaluated in a Discovery-3 rheometer (TA instruments, Australia) by oscillatory temperature ramps tested in the linear viscoelastic range. A stainless steel 40 mm parallel plate geometry was installed and gap-temperature compensation was performed for calibrations on the day of measurement and temperature was controlled by a single Peltier plate temperature system. CBD-distillate-infused sol-gel formulations (at 2-8° C.) were measured, with 0.5 mL of sample loaded onto the lower plate and 300 μm gap applied between parallel plates. A 300-second cycle of pre-conditioning at 8° C. was performed for each sol-gel sample. The solution-to-gel transition was recorded with a ramp setting of 5° C./minute up to 37° C. Results were estimated using a TRIOS software application.

To evaluate mucoadhesive strength, mucin discs were prepared first by compression of crude porcine mucin (120 mg) that were hydrated with 20 μL simulated nasal fluid (SNF) just before analysis. The mucoadhesive properties of sol-gel formulations were evaluated with a 4.5kg load cell using a Brookfield CT3 texture analyser. First, a mucin disc was fixed to the lower end of the instruments' probe with cyanoacrylate glue, then the formulation was filled into a beaker and equilibrated to 30±1° C. (target ceiling rheological gelation temperature). Next, the probe holding the mucosa was lowered on to the surface of the gel with a constant speed of 0.5 mm·s⁻¹ and a contact force of 0.06 N. The probe was then raised vertically upward at a constant speed of 0.5 mm·s⁻¹, with maximum force (the adhesive force, F) values then obtained from a force-distance correlation.

Three series of micelle powder compositions were prepared as set out in Examples 1 and 2 with varying amounts of Poloxamer P407, Tween® 80, propyl paraben and CBD distillate composition as set out in Table 1 below:

TABLE 1

| | P407 | T80 | PP | CBD- |
|---|---|---|---|---|
| Pre-formulation series | (% w/w) | (% w/w) | (% w/w) | distillate |
| Series A | 12 | 5 | 0.03 | 0.5 & 1 |
| Series B | 10 & 12 | 8 | 0.03 | 0.5 &1 |
| Series C | 12 | 8 | nil | 1 |

Compositions were then analysed for Zeta size, polydispersity and turbidity as set out in Tables 2 to 4 below.

TABLE 2
Series A CBD-distillate Micelles

| | Preformulation | | |
|---|---|---|---|
| Excipient (% w/w) | CB-COV-M1 | CB-COV-M2 | CB-COV-M3 |
| P407 | 12 | 12 | 12 |
| T80 | 5 | 5 | 5 |
| PP | Nil | 0.03 | 0.03 |
| CBD distillate | 1 | 1 | 0.5 |

Characteristics

| | | | |
|---|---|---|---|
| Zeta size (nm) | ND | 23.15 | 27.91 |
| PDI | ND | 0.03 | 0.21 |
| Turbidity (NTU) | ND | 2.56 | 1.7 |

ND = Not determined due to undesirable characteristics of the resultant sol-gel.

TABLE 3
Series B CBD-distillate Micelles

| | Pre-formulation | | | |
|---|---|---|---|---|
| | CB-COV- | CB-COV- | CB-COV- | CB-COV- |
| Excipients | M4 | M5 | M6 | M7 |
| P407 (% w/w) | 10 | 12 | 10 | 12 |
| T80 (% w/w) | 8 | 8 | 8 | 8 |
| PP (% w/w) | 0.03 | 0.03 | 0.03 | 0.03 |
| CBD distillate (% w/w) | 0.5 | 0.5 | 1 | 1 |

Pre-formulation characteristics

| | | | | |
|---|---|---|---|---|
| Zeta size (nm) | ND | ND | ND | 32.6 |
| PDI | ND | ND | ND | 0.19 |
| Turbidity (NTU) | ND | ND | ND | 1.94 |

ND = Not determined due to undesirable characteristics of the resultant sol-gel.

TABLE 4
Series C CBD-distillate Micelles
Stage 1 - Series C CBD-distillate micelles

| Pre-formulation | CB-COV-M8 |
|---|---|
| P407 (% w/w) | 12 |
| T80 (% w/w) | 8 |
| PP (% w/w) | nil |
| CBD distillate (% w/w) | 1.0 |

Pre-formulation characteristics

| | |
|---|---|
| Zeta size (nm) | 20.0 |
| PDI | 0.06 |
| Turbidity (NTU) | 1.62 |

Recorded turbidity values were <<10 NTU indicating visually undetectable particles upon dispersion in water, and within the accepted range for colloidal particles, i.e. 1-10 NTU. Micellar size was <<50 nm, which falls well within the desired size range (10-100 nm).

Sol-gel formulations were prepared with micelle formulations CB-COV-M2 to CB-COV-M8 and the sol-gels assessed for gelation state at cold temperature (2-8° C.), room temperature (22-24° C.) and nasal temperature (34-37° C.), gelation temperature, gel strength (Pa) at 8° C. and 30° C. and viscosity (Pa·s) at 8° C. and 30° C. The results are shown in Tables 5 to 7:

TABLE 5
Composition and characterisation of
sol-gels containing series A micelles

| | CB- | CB- | CB- | CB- |
|---|---|---|---|---|
| | COV- | COV- | COV- | COV- |
| Formulation | [symbol] | [symbol] | [symbol] | [symbol] |
| P407 (% w/w) | 0 | 0.5 | 1 | 1 |
| MP (% w/w) | 0.03 | 0.03 | 0.03 | 0.03 |
| CBD distillate (% w/w) | 1 | 1 | 1 | 0.5 |
| Micelle batch | M2 | M2 | M2 | M3 |

Gelation trend

| | | | | |
|---|---|---|---|---|
| CT | Liquid | Liquid | Liquid | Liquid |
| RT | Gel | Gel | Gel | Gel |
| NT | Gel | Gel | Gel | Gel |

Formulation characteristics

| | | | | |
|---|---|---|---|---|
| Gelation temp (° C.) | 15.8 | ND | ND | 22.8 |
| Gel Strength (Pa) at 8° C. | ND | ND | ND | ND |
| Gel Strength (Pa) at 30° C. | ND | ND | ND | ND |
| Viscosity (Pa · s) at 8° C. | ND | ND | ND | ND |
| Viscosity (Pa · s) at 30° C. | ND | ND | ND | ND |

ND—not determined as outside of desired gelation temperature range [symbol]
indicates data missing or illegible when filed

TABLE 6
Composition and characterisation of Sol-gels containing series B micelles

| Formulation | CB- | CB- | CB- | CB- | CB- | CB- | CB- | CB- | CB- | CB- |
|---|---|---|---|---|---|---|---|---|---|---|
| Excipients | COV- | COV- | COV- | COV- | COV- | COV- | COV- | COV- | COV- | COV- |
| P407 (% w/w) | 0 | 0.5 | 1 | 0 | 0.5 | 1 | 2 | 0.4 | 0.5 | 0.8 |
| MP (% w/w) | 0.03 | 0.03 | 0.03 | 0.03 | 0.03 | 0.03 | 0.03 | 0.03 | 0.03 | 0.03 |
| CBD distillate (% w/w) | 0.5 | 0.5 | 0.5 | 0.5 | 0.5 | 1 | 1 | 1 | 1 | 1 |
| Micelles batch | M4 | M4 | M4 | M5 | M5 | M6 | M6 | M7 | M7 | M7 |

Gelation trend

| | | | | | | | | | | |
|---|---|---|---|---|---|---|---|---|---|---|
| CT | Liquid | Liquid | Liquid | Liquid | Liquid | Liquid | Liquid | Liquid | Liquid | Liquid |
| RT | Liquid | Liquid | Gel | Liquid | Liquid | Liquid | Liquid | Liquid | Liquid | Liquid |
| NT | Liquid | Liquid | Gel | Gel | Gel | Liquid | Gel | Gel | Gel | Gel |

Formulation characteristics

| | | | | | | | | | | |
|---|---|---|---|---|---|---|---|---|---|---|
| Gelation temp (° C.) | ND | ND | 20.5 | 37.2 | 34 | ND | 37.2 | 35.9 | 35.8 | 30.2 |
| Gel Strength (Pa) at 8° C. | ND | ND | ND | ND | ND | ND | ND | ND | ND | 0.31 |
| Gel Strength (Pa) at 30° C. | ND | ND | ND | ND | ND | ND | ND | ND | ND | 1338.9 |
| Viscosity (Pa · s) at 8° C. | ND | ND | ND | ND | ND | ND | ND | ND | ND | 0.07 |
| Viscosity (Pa · s) at 30° C. | ND | ND | ND | ND | ND | ND | ND | ND | ND | 1.38 |

ND—not determined as outside of desired gelation temperature range

TABLE 7
Composition and characterisation of
Sol-gels containing series C micelles

| Formulation | CB-COV-SG15 |
|---|---|
| P407 (% w/w) | 0.8 |
| MP (% w/w) | nil |
| CBD distillate (% w/w) | 1 |
| Micelle batch | CB-COV-M8 |

Gelation trend

| | |
|---|---|
| CT | Liquid |
| RT | Liquid |
| NT | Gel |

Formulation characteristics

| | |
|---|---|
| Gelation temp (° C.) | 29.8 |
| Gel Strength (Pa) at 8° C. | 0.3 |
| Gel Strength (Pa) at 30° C. | 2601.8 |
| Viscosity (Pa · s) at 8° C. | 0.09 |
| Viscosity (Pa · s) at 30° C. | 1.62 |

The Sol-gel formulations containing the series A micelles resulted in premature gelation. Gelation at room temperature was not suitable for nasal delivery. In the series B micelles, the Tween® 80 was increased to 8% of the micelle composition to ensure solubility of the CBD in the highly concentrated distillate was maintained.

As shown in Table 6, small deviations in P407 concentration impact gelation behaviour, with the formulation CB-COV-SG14 prepared using 0.8% w/w P407 (Stage 2′ concentration only) showing a desirable gelation temperature (30.2° C.) alongside gel-strength (1338.9 Pa) and viscosity (1.38 Pa·s) at 30° C., as well as a clear visual appearance of the formulation at both room and nasal cavity temperature.

The preservative free Sol-gel formulation prepared with similar component amounts to CB-COV-SG14, also possessed desirable gelation and rheological characteristics.

### REFERENCES

Flores-Sanchez, I. J. and A. C. Ramos-Valdivia, A review from patents inspired by the genus Cannabis. Phytochemistry Reviews, 2017. 16(4): p. 639-675.
Hanus, L. O., et al., Phytocannabinoids: a unified critical inventory. Nat Prod Rep, 2016. 33(12): p. 1357-1392.
ElSohly, M. A., et al., Phytochemistry of Cannabis sativa L. Prog Chem Org Nat Prod, 2017. 103: p. 1-36.
Mackie, K., Cannabinoid Receptors: Where They are and What They do. Journal of Neuroendocrinology, 2008. 20(s1): p. 10-14.
Zou, S. and U. Kumar, Cannabinoid Receptors and the Endocannabinoid System: Signaling and Function in the Central Nervous System. International journal of molecular sciences, 2018. 19(3): p. 833.
De Vita, M. J., et al., Association of Cannabinoid Administration With Experimental Pain in Healthy Adults: A Systematic Review and Meta-analysis. JAMA Psychiatry, 2018. 75(11): p. 1118-1127.
Andreae, M. H., et al., Inhaled Cannabis for Chronic Neuropathic Pain: A Meta-analysis of Individual Patient Data. The Journal of Pain, 2015. 16(12): p. 1221-1232.
Koppel, B. S., et al., Systematic review: efficacy and safety of medical marijuana in selected neurologic disorders: report of the Guideline Development Subcommittee of the American Academy of Neurology. Neurology, 2014. 82(17): p. 1556-63.

1.-26. (canceled)

27. A thermoresponsive sol-gel formulation comprising
a) a thermoresponsive poloxamer and/or poloxamine and
b) a micellar composition comprising a plurality of micelles, said micelles comprising:
i) thermoresponsive poloxamer and/or poloxamine;
ii) surfactant in an amount of 7-9% w/w of the sol-gel formulation, wherein the surfactant is not a poloxamer and/or poloxamine; and
iii) a cannabinoid composition in an amount of 0.5 to 5 w/w of the sol-gel formulation;
wherein the sol-gel formulation has total amount of thermoresponsive poloxamer of 12 to 13.5% w/w of the sol-gel formulation, a gelation temperature between 28° C. and 32° C. and the cannabinoid composition comprises at least 70% w/w of cannabidiol.

28. The sol-gel formulation according to claim 27 wherein the micellar composition comprises about 11.8 to 12.2% w/w thermoresponsive poloxamer and/or poloxamine based on the sol-gel formulation.

29. The sol-gel formulation according to claim 27 where in the thermoresponsive poloxamer and/or poloxamine of part a) is present in an amount of about 0.8% w/w of the sol-gel formulation.

30. The sol-gel formulation according to claim 27 wherein the thermoresponsive poloxamer in the micellar composition and/or in part a) is P407.

31. The sol-gel formulation according to claim 27 wherein the surfactant in the micellar composition is polyoxyethylene sorbitan monooleate.

32. The sol-gel formulation according to claim 27 wherein the cannabinoid composition:
a. is present in an amount of about 1% of the sol-gel formulation; and/or
b. comprises 75 to 90% w/w CBD, or about 80% w/w CBD.

33. The sol-gel formulation according to claim 27 having a gelation temperature between 29° C. and 31° C.

34. The sol-gel formulation according to claim 27 having a viscosity of:
a. less than 0.15 Pa·s at about 8° C.; and
b. greater than about 1.0 Pa·s at about 30° C.

35. The sol-gel formulation according to claim 27 wherein the sol-gel formulation has a gel strength of greater than about 1000 Pa at about 30° C.

36. The sol-gel formulation according to claim 27 further comprising one or more preservatives.

37. The sol-gel formulation according to claim 36 wherein:
a. the micellar composition comprises a preservative; and/or
b. part a) of the formulation comprises a preservative.

38. The sol-gel formulation according to claim 37 wherein the preservative in the micellar composition is propyl paraben; and/or wherein the preservative in part a) of the formulation is methyl paraben.

39. A thermoresponsive sol-gel formulation comprising
a) P407 in an amount of about 0.8% w/w of the sol-gel formulation and
b) a micellar composition comprising a plurality of micelles, said micelles comprising:
i) P407 in an amount of about 12% w/w of the sol-gel formulation;
ii) polyoxyethylene sorbitan monooleate in an amount of 7-9% w/w of the sol-gel formulation; and
iii) a cannabinoid composition in an amount of 0.5 to 1.2% w/w of the sol-gel formulation;
wherein the sol-gel formulation has total amount of P407 of about 12.8% w/w of the sol-gel formulation, a gelation temperature between 29° C. and 31° C. and the cannabinoid composition comprises at least 75% w/w of cannabidiol.

40. A method of treating or preventing or alleviating one or more symptoms of a Coronavirus infection comprising administering a thermoresponsive sol-gel formulation according to claim 27.

41. The method according to claim 40, wherein the Coronavirus infection is SARS-COV-2.

42. The method according to claim 40, wherein the thermoresponsive sol-gel formulation is administered to the nasal cavity.

43. A method of treating or preventing or alleviating one or more symptoms of a Coronavirus infection comprising administering a thermoresponsive sol-gel formulation according to claim 39.

44. The method according to claim 43, wherein the Coronavirus infection is SARS-COV-2.

45. The method according to claim 43, wherein the thermoresponsive sol-gel formulation is administered to the nasal cavity.
